(19) United States
(12) Patent Application Publication
(10) Pub. No.: US 2024/0000456 A1
(43) Pub. Date: Jan. 4, 2024

(54) CLOSURE DEVICES, VASCULAR REPAIR ASSEMBLIES, AND METHODS FOR REPAIRING VEIN VALVE INSUFFICIENCY

(71) Applicant: Bard Peripheral Vascular, Inc., Franklin Lakes, NJ (US)

(72) Inventors: Huffer Katherine, Chandler, AZ (US); Kelly Joann, Tempe, AZ (US)

(73) Assignee: Bard Peripheral Vascular, Inc., Franklin Lakes, NJ (US)

(21) Appl. No.: 18/245,046

(22) PCT Filed: Sep. 15, 2020
(86) PCT No.: PCT/US2020/050890

§ 371 Date: Mar. 13, 2023

(51) Int. Cl. A61B 17/12

(52) U.S. Cl. CPC A61B 17/12109; A61B 2017/00778

(57) ABSTRACT

A closure device for repairing a vein valve insufficiency includes a tube formed of extracellular matrix including elastin fibers and one or more anchoring elements. The tube is radially expandable from a retracted configuration to an expanded configuration and the tube is naturally biased to the retracted configuration. The one or more anchoring elements anchor the tube to a vessel wall of a vessel upon expansion of the tube to the expanded configuration wherein the tube circumferentially contacts the vessel wall of the vessel and is anchored thereto by the one or more anchoring elements. Retraction of the tube to the retracted configuration draws the vessel wall of the vessel radially inward to repair the vein valve insufficiency.

### TECHNICAL FIELD

The present specification generally relates to closure devices, vascular repair assemblies, and methods for repairing vein valve insufficiency and, more specifically, closure devices vascular repair assemblies, and methods for repairing vein valve insufficiency including a radially expandable tube, which may be anchored within a vessel to drawing the vessel to a smaller diameter from within.

### BACKGROUND

A healthy vein valve functions to prevent retrograde flow of blood and allow only antegrade flow of blood to the heart. An incompetent vein valve (also known as an insufficient vein valve or a leaky vein valve) may not seal properly and may allow retrograde flow. Incompetence of a venous valve is thought to arise from varicose veins, chronic venous insufficiency, or the like. In some cases, insufficient venous valves may result from surgeries, where portions of vein may be expanded (such as in blood clot removal). FIG. 1A depicts healthy venous valve 12 of a vessel 10 (e.g., a vein). The valve is bicuspid, with opposed cusps or leaflets 14a, 14b. In the closed condition, the leaflets 14a, 14b are drawn together to prevent retrograde flow of blood. Referring to FIG. 1B, if the valve is incompetent, the leaflets 14 do not seal properly and retrograde flow of blood occurs. It is noted that devices which may replace vein valves or repair vein valves in the aortic region may not the applicable to restoring natural vein valve functionality within the arms and/or legs of a patient as they might not be sized, shaped, or structured to facilitate repairing vein valves at locations outside of the aortic region. For example, a vein may be delicate compared to aortic regions, with a greater potential of tearing. Accordingly, devices used to repair or replace aortic valves may not be applicable to venous valves.

### SUMMARY

Embodiments of the present disclosure are directed to improvements over the above limitations by providing closure devices, vascular repair assemblies, and methods for restoring natural vein valve functionality within regions of the body such as the arms and/or legs of the patient, though other regions are contemplated and possible.

In one embodiment, a closure device for repairing a vein valve insufficiency includes a tube and one or more anchoring elements. The tube is formed of extracellular matrix including elastin fibers and is radially expandable from a retracted configuration to an expanded configuration, wherein the tube is naturally biased to the retracted configuration. The one or more anchoring elements are configured to anchor the tube to a vessel wall of a vessel upon expansion of the tube to the expanded configuration wherein the tube circumferentially contacts the vessel wall of the vessel and is anchored thereto by the one or more anchoring elements. Retraction of the tube to the retracted configuration draws the vessel wall of the vessel radially inward to repair the vein valve insufficiency.

In another embodiment, a vascular repair assembly includes an expandable balloon, wherein the expandable balloon is configured to radially expand, and a closure device removably mounted to the expandable balloon for delivery into a body lumen to be repaired. The closure device includes a tube, and one or more anchoring elements. The tube is formed of extracellular matrix including elastin fibers. The expandable balloon is positioned within a lumen of the tube and the tube is radially expandable from a retracted configuration to an expanded configuration in response to expansion of the expandable balloon. The tube is naturally biased to the retracted configuration upon removal of the expandable balloon. The one or more anchoring elements are configured to anchor the tube to a vessel wall of the vessel upon expansion of the tube to the expanded configuration wherein the tube circumferentially contacts the vessel wall of the vessel and is anchored thereto by the one or more anchoring elements. Deflation of the expandable balloon allows the tube to retract to the retracted configuration thereby drawing the vessel wall of the vessel radially inward as the tube retracts to the retracted configuration

In yet another embodiment, a method for repairing a vein valve insufficiency includes advancing a closure device mounted to an expandable balloon through a body lumen to a position upstream of a target vein valve, wherein the closure device comprises a tube and one or more anchoring elements, the tube being formed of extracellular matrix comprising elastin fibers, expanding the tube to an expanded configuration wherein the tube is in radial contact with a vessel wall of the vessel and the one or more anchoring elements anchor the tube to the vessel wall of the vessel, and deflating the expandable balloon, wherein deflation of the expandable balloon allows the tube to retract to a retracted configuration, wherein the tube is naturally biased to the retracted configuration.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically illustrates a functional vein valve;

FIG. 1B schematically depicts an incompetent vein valve;

FIG. 2 schematically depicts a closure device, according to one or more embodiments shown and described herein;

FIG. 3 schematically depicts a cross-sectional view of a closure device, according to one or more embodiments shown and described herein;

FIG. 4A schematically depicts a closure device in a low-profile delivery configuration, according to one or more embodiments shown and described herein;

FIG. 4B schematically depicts the closure device of FIG. 4A in an expanded tissue-engaging configuration, according to one or more embodiments shown and described herein;

FIG. 5A schematically depicts a cross-sectional view of a closure device in a low-profile delivery configuration, according to one or more embodiments shown and described herein;

FIG. 5B schematically depicts the closure device of FIG. 5A in an expanded tissue-engaging configuration, according to one or more embodiments shown and described herein;

FIG. 6A schematically depicts a partial top view of one or more guide paths formed within a tube of a closure device, according to one or more embodiments shown and described herein;

FIG. 6B schematically illustrates a side view of a retention member of the closure device positioned below the one or more guide paths formed in the tube of FIG. 6A, according to one or more embodiments shown and described herein;

FIG. 6C schematically depicts the retention member of FIG. 6B traversing the guide paths formed in the tube, according to one or more embodiments shown and described herein;

FIG. 6D schematically depicts the retention member of FIG. 6C anchoring the tube of the closure device to a vessel wall of a vessel, according to one or more embodiments shown and described herein;

FIG. 7A schematically depicts a partial top view of a guide path formed within a tube of a closure device, according to one or more embodiments shown and described herein;

FIG. 7B schematically illustrates a side view of a retention member of the closure device positioned partially within the guide path formed in the tube of FIG. 7A, according to one or more embodiments shown and described herein;

FIG. 7C schematically depicts the retention member of FIG. 7B anchoring the tube of the closure device to a vessel wall of a vessel, according to one or more embodiments shown and described herein;

FIG. 8A schematically depicts yet another embodiment of a retention member positioned below a guide path formed in a tube of a closure device, according to one or more embodiments shown and described herein;

FIG. 8B schematically depicts the retention member of FIG. 8A traversing the guide path, according to one or more embodiments shown and described herein;

FIG. 8C schematically depicts an expandable portion of the retention member of FIG. 8B expanding to constrain the tube to a vessel wall of a vessel, according to one or more embodiments shown and described herein;

FIG. 8D schematically depicts a perspective view of the retention member of FIG. 8C anchoring the tube to the vessel wall of the vessel, according to one or more embodiments shown and described herein;

FIG. 9 schematically depicts a vascular repair assembly including a closure device, according to one or more embodiments shown and described herein;

FIG. 10A schematically depicts a vascular repair assembly positioned within a vessel of a subject, according to one or more embodiments shown and described herein;

FIG. 10B schematically a cross-sectional view of the vascular repair assembly of FIG. 10A in a low-profile delivery orientation, according to one or more embodiments shown and described herein;

FIG. 10C schematically depicts a cross-sectional view of the vascular repair assembly of FIG. 10A expanded via an expansion device to an expanded configuration so as the engage the vessel to anchor a closure device of the vascular repair assembly to the vessel wall of the vessel, according to one or more embodiments shown and described herein;

FIG. 10D schematically depicts the expansion device of FIG. 10C retracted back to the low-profile delivery configuration thereby separating the expansion device from the closure device, according to one or more embodiments shown and described herein;

FIG. 10E schematically depicts retraction of the closure device while anchored to the vessel wall such that the vessel wall is retracted along with the closure device to a narrower diameter, according to one or more embodiments shown and described herein;

FIG. 11 schematically depicts another embodiment of a vascular repair assembly deployed in a vessel, according to one or more embodiments shown and described herein;

FIG. 12 schematically depicts yet another vascular repair assembly deployed in a vessel, according to one or more embodiments shown and described herein;

FIG. 13 depicts a flow chart illustrating a method for repairing vein valve insufficiency according to one or more embodiments shown and described herein; and

FIG. 14 depicts a flow chart illustrating a method for assembling a vascular repair assembly according to one or more embodiments shown and described herein.

### DETAILED DESCRIPTION

Embodiments of the present disclosure are directed to closure devices, vascular repair assemblies, and methods for repairing a vein valve insufficiency. For example, a closure device may include a tube formed of extracellular matrix, having elastin fibers, wherein the tube is radially expandable from a retracted configuration to an expanded configuration. The tube is formed so as to be naturally biased to the retracted configuration. One or more anchoring elements are configured to anchor the tube to a vessel wall of a vessel (e.g., a vein or other bodily lumen) upon expansion of the tube to the expanded configuration. When expanded, the tube circumferentially contacts the vessel wall of the vessel and is anchored thereto by the one or more anchoring elements. The tube may be retracted to the retracted configuration thereby drawing the vessel wall of the vessel radially inward to repair the vein valve insufficiency. Accordingly, the tube may pull the vessel wall back to an operable diameter to restore natural function to the vessel valve. It is noted that in some embodiments, the devices, assemblies, and methods as described herein may be used to draw in or reduce a diameter of other bodily vessels or portions thereof (e.g., veins, arteries, organs, etc.) to a desired diameter. Various embodiments will now be described in greater detail below with reference to the figures.

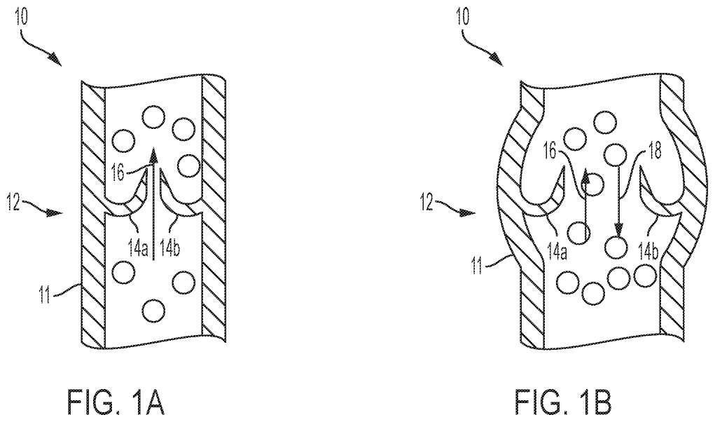

As described above, FIGS. 1A and 1B depict a vessel 10, such as a vein, though other body vessels are contemplated and possible without departing from the scope of the present disclosure. The vessel 10 includes a vessel wall 11 and a valve 12, such as a vein valve. The valve 12 includes leaflets 14a, 14b configured to move between an open position and closed position. In particular, a functional vein valve 12, such as illustrated in FIG. 1A, is configured to open and close as blood is pumped through the body. For example, as the blood is pumped through the body, the valve leaflets 14a, 14b open in response to blood pushing past the leaflets 14a, 14b. In between pumps of the heart, the leaflets 14a, 14b close to prevent blood from flowing backward through the valve 12. Accordingly, blood only flows through the valve 12 in the downstream direction 16. However, as illustrated in FIG. 1B, the leaflets 14a, 14b are positioned too far apart to be able to close, thereby allowing blood to both in the downstream direction 16 and the upstream direction 18, also known a regurgitation, making it difficult to return blood to the heart. Embodiments as described herein includes devices, assemblies, and methods configured to be deployed within the vessel at a position adjacent the valve 12 to pull the vessel to a smaller diameter to restore the natural function of the valve 12.

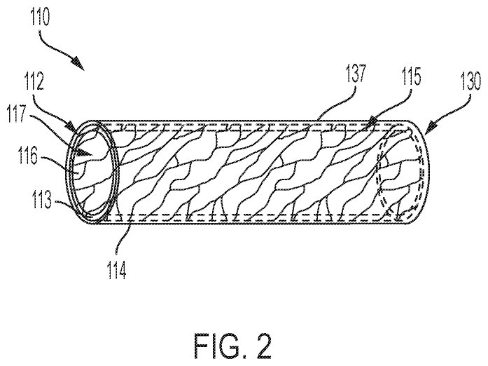

Referring now to FIG. 2, a closure device 110 is schematically depicted. As will be described in greater detail herein, the closure device is configured to be stretched or dilated to circumferentially engage with a vessel wall 11 and then retracted to pull the vessel wall 11 to a smaller diameter. That is, the closure device 110 may be configured to be inserted into a vessel 10 proximate to an incompetent vein valve, for example, and cause the diameter of the vein to be reduced in the region of the incompetent vein valve, thereby restoring the natural function to the vein valve (i.e., pulling the leaflets 14a, 14b into close enough proximity with one another to allow the leaflets to functionally move between the open and closed positions thereby preventing regurgitation or backflow through the valve 12).

In embodiments, the closure device 110 includes a tube 112 formed of extracellular matrix (“ECM”). As used herein, the terms “extracellular matrix” and “ECM” refer to a complex mixture of structural and/or functional biomolecules including, but not limited to, structural proteins, specialized proteins, proteoglycans, glycosaminoglycans, growth factors, or any combination thereof, that surround and support cells within mammalian tissues. ECM may be obtained from various donor organs and tissues (such as human, bovine, porcine, ovine or similar tissues). For example, ECM may be derived from small intestinal submucosa. In some embodiments, the ECM may be decellularized using various techniques, for example the chemical, enzymatic, or mechanical disruption. At least a portion of the ECM of the tube 112 includes elastin fibers 115. For example, 20% or more by weight, 30% or more by weight, 40% or more by weight, 50% or more by weight, 60% or more by weight, 70% or more by weight, 80% or more by weight, 90% or more by weight, or the like. A greater amount of elastin fibers 115 may provide a greater pulling force for pulling the tube to a retracted diameter. The elastin fibers 115 may be elongate fibers having a length that is greater than a width or diameter of the elastin fibers 115. For example fiber diameters include, but are not limited to, about 1 micron to about 6 microns.

The tube 112 may generally define an elongated body having a tube wall 113 defining an outer surface 114 and an inner surface 116, wherein the inner surface 116 defines a lumen 117 extending through the tube 112. As will be described in greater detail herein, the lumen 117 is sized to receive an expansion device therein such that the tube 112 may be radially expanded to circumferentially contact a vessel wall. To form the tube 112, the ECM including the elastin fibers 115 may be formed around a mold (e.g., a cylindrical mold) to provide a tube-like structure or may be grown around a removable cylindrical structure. The elastin fibers 115 may be arranged within the tubular shape of the tube 112 and allow the tube 112 to diametrically and elastically stretch and retract. For example, a diameter of the tube 112 may be configured to stretch to at least 50% larger than its original diameter, at least 80% larger than its original diameter, at least 100% larger than its original diameter, at least 130% larger than its original diameter, at least 150% larger than its original diameter, or the like. Upon release, the tube 112, the elastin fibers 115 may cause the tube 112 to naturally retract back to its original diameter or a retracted diameter that is smaller than an expanded diameter. Accordingly, the diameter of the tube 112 may be chosen based on a desired diameter of the vessel. For example, a retracted diameter of the tube 112 may be configured to restore vein valve functionality within the particular vessel.

The closure device 110 may further include one or more anchoring elements 130 configured to anchor the tube 112 of the closure device 110 to the vessel wall 11 of the vessel 10 when expanded into contact with the vessel wall 11 of the vessel 10. The following description details embodiments of various, non-limiting, anchoring elements 130 which may be used to anchor the tube 112 of the closure device 110 to the vessel wall 11. Accordingly, the above description of the tube 112 of the closure device 110 is applicable to each of the embodiments described herein, unless otherwise noted. It is noted that though various anchoring elements 130 are illustrated as being used in isolation from other types of anchoring elements, in embodiments, one or more of the various anchoring elements 130 may be used simultaneously with one another.

Still in reference to FIG. 2, in some embodiments, the one or more anchoring elements 130 may include an adhesive 137 (e.g., a bio-compatible adhesive and/or a cell adhesion molecule such as, but not limited to immunoglobulin cell adhesion molecules (IgCAMs), Cadherin, Integrins, C-type of lectin-like domains proteins (CTLDs), proteoglycans, or the like). As illustrated in FIG. 2, the adhesive 137 may be formed and/or coated on an outer surface 114 of the tube 112 of the closure device 110 such that the tube 112 may become adhered (e.g., via a chemical reaction between the adhesive 137 and the vessel wall 11) to the vessel wall 11 when positioned in contact with the vessel wall 11. The adhesive 137 may be coated over the entire outer surface 114 of the tube 112 or only a portion thereof.

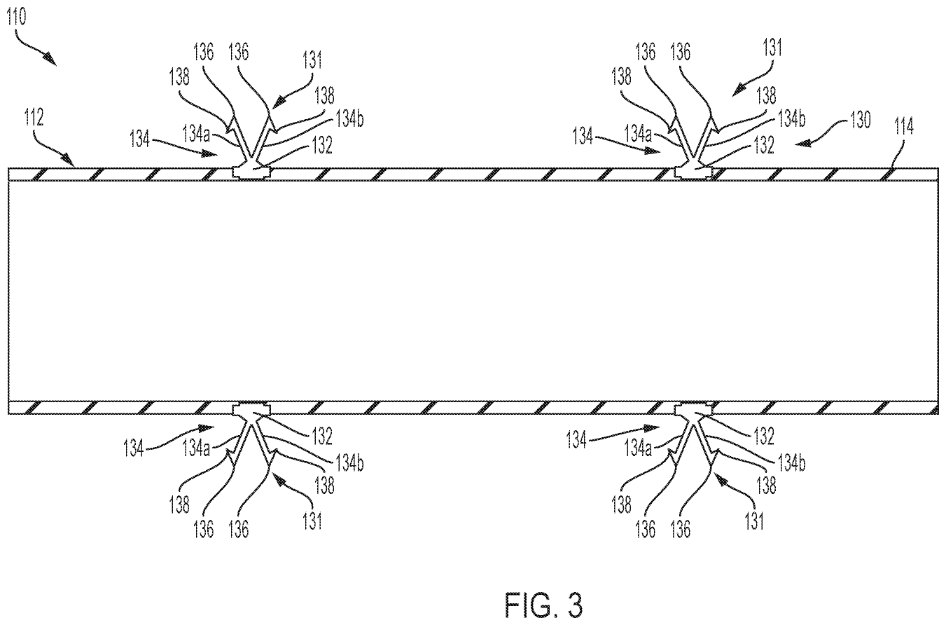

Referring now to FIG. 3, a longitudinal cross-section of an embodiment of a closure device 110 is schematically depicted. In the illustrated embodiment, the one or more anchoring elements 130 includes a plurality of retention members 131 coupled to the tube 112 and/or configured to be coupled to the tube 112. The plurality of retention members 131 may include any hook-like structures configured to pierce the vessel wall 11 of the vessel 10 thereby becoming anchored thereto. For example, each of the retention members 131 may include a base 132 coupled to the tube 112. For example, the base 132 may be bonded, adhered of fastened to the tube 112. In some embodiments, the ECM of the tube 112 may be formed or grown around the retention members 131 thereby coupling the retention members 131 to the tube 112. In some embodiments, the base 132 may be embedded within the tube wall 113 of the tube 112.

Extending from the base 132 may be one or more retention legs 134 (e.g., such as two or more retention legs, three or more retention legs, four or more retention legs, etc.). For example, in the illustrated embodiment, the one or more retention legs 134 include a first retention leg 134a and a second retention leg 134b. Each of the one or more retention legs 134 may have a sharp distal tip 136 configured to pierce the vessel wall 11 and a hook 138 configured couple the plurality of retention members 131 to the vessel wall 11. The plurality of retention members 131 including the base 132 and the one or more retention legs 134 may be formed of ECM, biocompatible metals/metal alloys (such as nitinol, stainless steel, or the like), biocompatible polymers, or any combination thereof. In embodiments, the base 132 and the one or more retention legs 134 may be integrally formed (e.g., molded). In other embodiments, one or more retention legs 134 may be bonded, welded, soldered, fastened, or the like to the base 132.

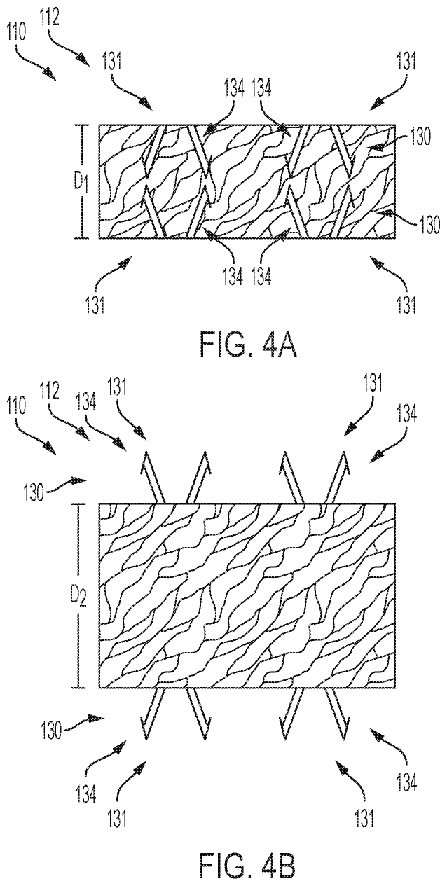

In some embodiments, the plurality of retention members 131 may have a low-profile delivery configuration and an expanded tissue-engaging configuration. For example, and with reference to FIGS. 4A and 4B an embodiment of a closure device 110 is schematically depicted. In FIG. 4A, the closure device 110 is illustrated in a low-profile delivery configuration having a first diameter D₁. While in the low-profile delivery configuration, the plurality of retention members 131, in particular, the one or more retention legs 134 may be folded or positioned against the tube 112. Referring now to FIG. 4B, as the tube 112 is expanded to a second diameter D₂, which is larger than the first diameter, the one or more retention legs 134 may be biased or moved to unfold or straightened so as to be positioned to pierce a vessel wall 11.

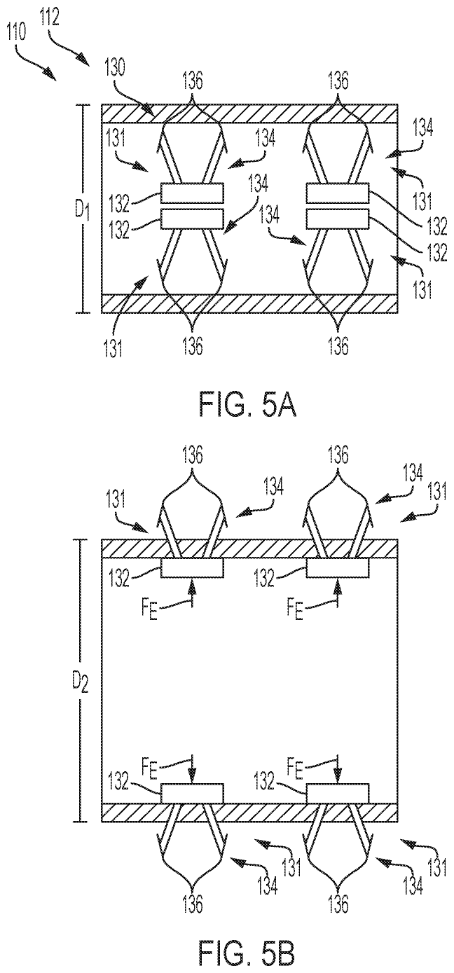

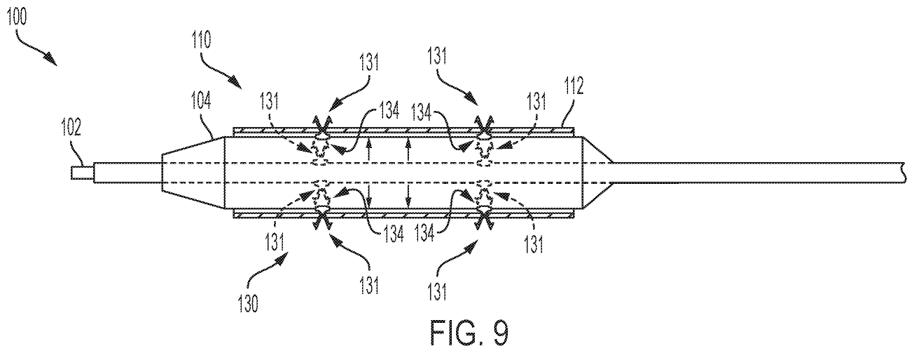

Referring now to FIGS. 5A and 5B, in some embodiments, the plurality of retention members 131 may instead be placed radially within the tube 112 in the low-profile delivery configuration and may be moved to extend through the tube 112 in the expanded tissue-engaging configuration. The may aid in traversing the closure device 110 through the vessel 10 of the subject without catching the wall of the vessel 10 prior to reaching to point of deployment. With reference to FIG. 5A the closure device 110 is depicted in the low-profile delivery configuration having a diameter D₁. While in the low-profile delivery configuration, the plurality of anchoring elements 130 may be fully or at least partially positioned within the tube 112. During expansion to the second diameter D₂, the plurality of anchoring elements 130 may be pushed via an expansion force FE (e.g., provided via an expansion device 104, such as schematically depicted in FIG. 9) radially outward through the tube 112 to allow the one or more retention legs 134 to pierce a vessel wall 11. In such embodiments the tube 112 may be sandwiched between the base 132 of each retention member 131 and the vessel wall 11 when anchored to the vessel wall 11. In such embodiments, the sharp distal tips 136 may pierce the tube wall 113 of the tube 112 during expansion from the low-profile delivery configuration (illustrated in FIG. 5A) to the expanded tissue-engaging configuration (FIG. 5B), or there may be openings or guide paths through the tube wall 113 through which the one or more retention legs 134 extend.

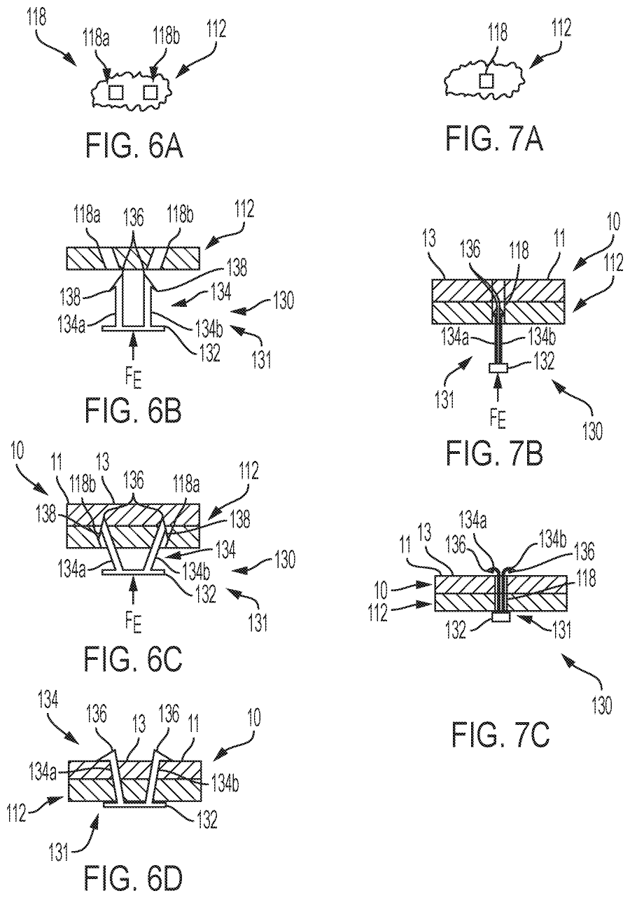

FIGS. 6A-6D illustrate, an embodiment wherein a retention member 131 is first positioned within the tube 112 in a low-profile delivery configuration and then expanded to an expanded tissue-engaging configuration via application of an expansion force FE. Referring to FIG. 6A, a partial top view of the tube 112 is schematically illustrated. Formed within the tube 112 may be one or more guide paths 118. The one or more guide paths 118 may be configured to receive the one or more retention legs 134 of the retention member 131 to guide the one or more retention legs 134 through the tube 112 and into the vessel wall 11. The number of the one or more guide paths 118 may be equal to the number of retention legs 134 of each retention member 131. For example, wherein the retention member 131 includes a first retention leg 134a and a second retention leg 134b, there may be a corresponding first guide path 118a and second guide path 118b. In the illustrated embodiment, the first retention leg 134a and the second retention legs 134b may extend from the base 132 at a first orientation. For example, the first retention leg 134a and the second retention leg 134b may be substantially parallel to one another, as illustrated in FIG. 6B. As the expansion force FE is applied to the base 132 to expand the closure device 110, the first retention leg 134 and the second retention leg 134b may traverse the first guide path 118a and the second guide path 118b respectively. The first guide path 118a and the second guide path 118b may be angled with respect to one another at some non-parallel angle such that traversal of the first retention leg 134a and the second retention leg 134b causes the first retention leg 134a and the second retention leg 134b to diverge from one another as the first retention leg 134a and the second retention leg 134b are advanced through the first guide path 118a and the second guide path 118b, respectively, as illustrated in FIG. 6C. As illustrated in FIG. 6D, the sharp distal tips 136 of the first retention leg 134 and the second retention leg 134b may be advanced through the vessel wall 11 such that the hooks 138 engage an outer surface 13 of the vessel wall 11 thereby anchoring the tube 112 to the vessel wall 11. In such embodiments, the tube 112, and the vessel wall 11 may be sandwiched between the base 132 and the hooks 138 of the retention member 131.

In some embodiments, the first retention leg 134a and the second retention leg 134b may be naturally biased to diverge from one another. For example, the first retention leg 134a and the second retention leg 134b may be formed of a shape memory material that is configured to bend as it is advanced out of the tube 112. In such embodiments, the one or more guide paths 118 may include a single guide path 118 through the tube 112, such as illustrated in FIG. 7A. That is, each of the first retention leg 134 and the second retention leg 134b may be advanced together through the same guide path 118. However, separate guide paths 118 such as illustrated in FIGS. 6A-6D are contemplated and possible. In some embodiments, the sharp distal tip 136 of the first retention leg 134a and the second retention leg 134b may be positioned within the guide path 118 to restrain the first retention leg 134a and the second retention leg 134b from diverging prior to exiting the guide path 118, as illustrated in FIG. 7B. As the expansion force FE is applied to the base 132 of the anchoring element 130 to expand the closure device 110 from the low-profile delivery configuration to the expanded tissue-engaging configuration, the first and second retention legs 134a, 134b may traverse the guide path 118 and pierce the vessel wall 11. As the first and second retention legs 134a, 134b pierce through the vessel wall 11, the first and second retention legs 134a, 134b may be biased to diverge from one another so as to extend over the outer surface 13 of the vessel 10 such that the tube 112 and the vessel wall 11 are sandwiched between the hooks 138 of the first and second retention legs 134 and the base 132.

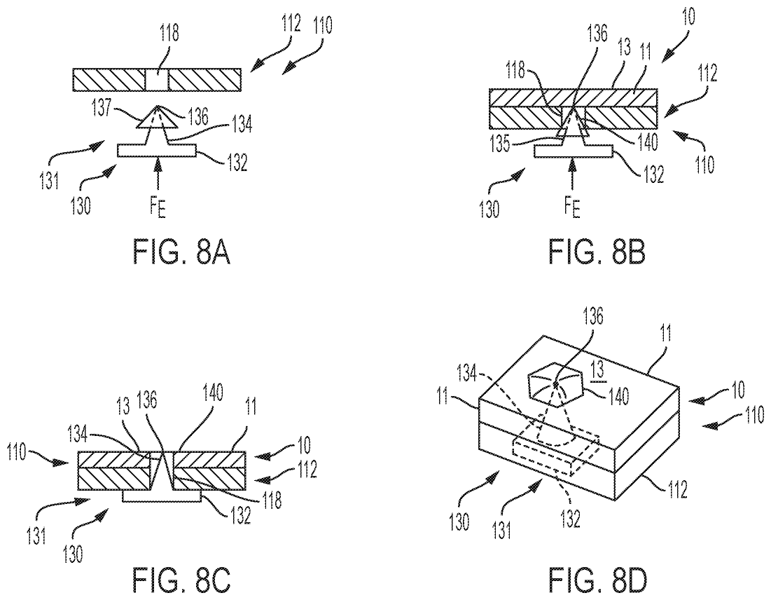

Referring now to FIGS. 8A-8D, yet another embodiment of a retention member 131 is depicted. In the present embodiment, the retention member 131 includes a retention leg 134 having a sharp distal tip 136 (e.g., needle-like tip, or any tip suitable for piercing a vessel wall), similar to the embodiments described above. The base 132 is coupled to a proximal end of the retention leg 134 opposite the sharp distal tip 136. In the depicted embodiment, the retention leg 134 has an increasing diameter from the sharp distal tip 136 to the base 132 so as to be substantially cone-shaped, for example, though other shapes are contemplated and possible (e.g., pyramid, tetrahedron, or the like).

Coupled to the sharp distal tip 136 (e.g., via adhesive, welding, or the like) may be an expandable shield 140. The expandable shield 140 may be formed of folded material (e.g., ECM, nitinol, etc.) that has that may be compressed at it is traversed through a guide path 118 formed in the tube 112 as the expansion force FE is applied to the retention member 131, as illustrated in FIG. 8B. Upon passing through guide path 118 of the tube 112 and the vessel wall 11 of the vessel 10, the expandable shield 140 may expand as illustrated in FIGS. 8C and 8D. For example, the expandable shield 140 may radially be biased to expand around the sharp distal tip 136 once the expandable shield 140 extends across outer surface 13 the vessel wall 11. For example, and not as a limitation, in the expanded state, the expandable shield 140 may be round, oval, hexagonal, octagonal, or any polygonal or non-polygonal shape. Accordingly, the vessel wall 11 and the tube 112 may be sandwiched between the expandable shield 140 and the base 132, thereby anchoring the tube 112 to the vessel 10.

It is noted that in the above embodiments, various portions of the retention members 131 may extend completely through the vessel wall 11 of the vessel 10. However, in some embodiments, the retention members 131 may only extend through a portion of the vessel wall 11 of the vessel 10, thereby anchoring the closure device 110 within the vessel wall 11 of the vessel 10.

It is noted that in the above embodiments, any number of retention members 131 (two or more, four or more, six or more, etc.) may be included without departing from the scope of the present disclosure. For example, a plurality of retention members 131 may be arranged around various radial positions of the tube 112. In some embodiments, at least some of the retention members 131 may be diametrically opposed to one another such as illustrated in FIGS. 3-5B. It is further noted that though several distinct retention members 131 are depicted in the figures, other variations are contemplated and possible. Moreover, different types of retention members 131 may be used in conjunction with one another. In yet further embodiments, the one or more anchoring elements 130 may include a combination of retention members 131 and adhesive 137, as described above.

Referring now to FIG. 9 a vascular repair assembly 100 is schematically depicted. The vascular repair assembly 100 generally includes a catheter 102, an expansion device 104, and a closure device 110 according to any of the embodiments described herein.

For example, catheter 102 may include any type of flexible tubing configured for traversal through one or more body vessels. In particular, the catheter 102 may be sized and shaped to be traversed through a vein of a user to a location of a dysfunctional vein valve, as described above. Mounted to the catheter 102 may be an expansion device 104 configured to radially expand around the catheter 102. The expansion device 104 may be a balloon (such as an angioplasty balloon), an expandable cage, stent, stent graft, or other similar device configured to radially expand about the catheter 102. In some embodiments, the expansion device 104 may be integrated into the catheter 102 such as in a balloon catheter. The closure device 110, according to any of the embodiments described herein, may be mounted to the expansion device 104 such that when the expansion device 104 radially expands about the catheter 102, the closure device 110 also radially expands about the catheter 102.

As noted in the embodiments above, in embodiments where the one or more anchoring elements 130 include retention members 131, such as described above, the retention members 131 may be mounted within the tube 112 prior to expansion to the expanded tissue-engaging configuration. As noted herein, application of an expansion force FE, provided via the expansion device 104, may drive the retention members 131 through the tube wall 113 of the tube 112 to engage the vessel wall 11 of the vessel 10, thereby anchoring the closure device 110 to the vessel wall 11. In some embodiments, the base 132 or a portion of the base 132 of the retention member 131 may be mounted to the expansion device 104. For example, the base 132 may be removably mounted to the expansion device 104 (e.g., via an adhesive, mechanical coupling, or the like). In some embodiments, and as will be described in more detail below, the portion of the base 132 attached to the expansion device 104 may break away upon retraction of the expansion device 104.

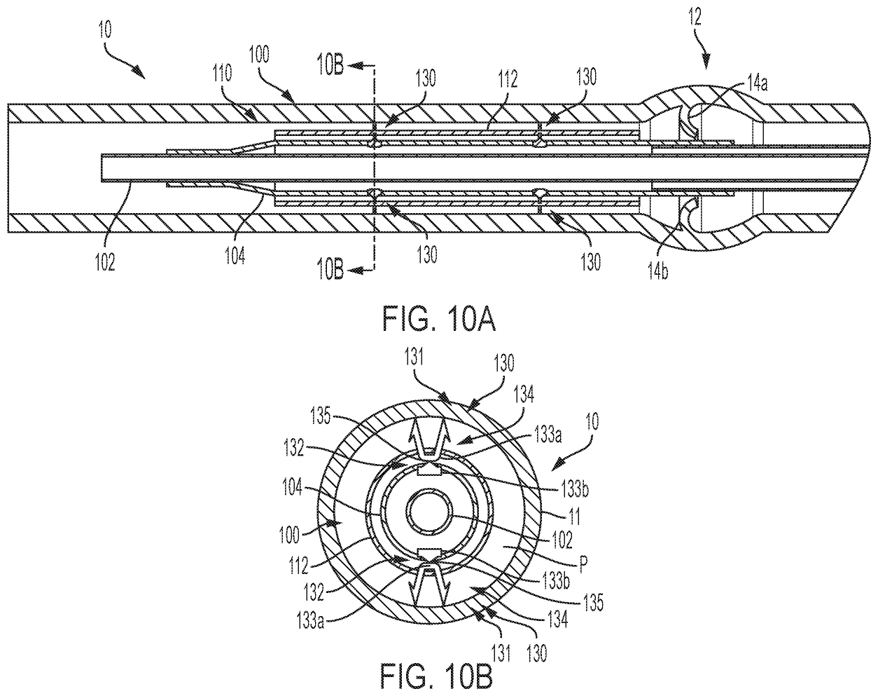

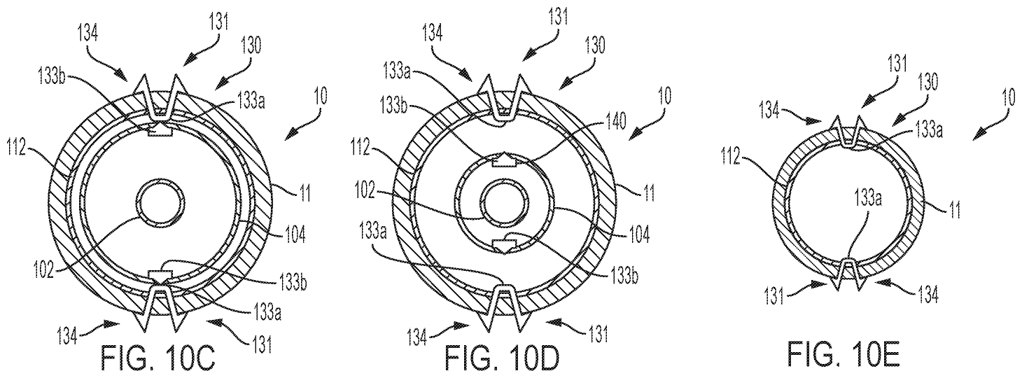

Referring now to FIG. 10A, a vascular repair assembly 100 according to one or more embodiments described herein, includes the catheter 102, the expansion device 104, and the closure device 110 are illustrated within a vessel 10. The vascular repair assembly 100 may have been advanced from an access site, not shown through the vasculature of the subject to a desired position (e.g., adjacent an incompetent valve 12). For example, the vascular repair assembly 100 may have been advanced through the valve 12 in the upstream direction 18 such that the closure device 110 is positioned adjacent the valve 12. However, it is contemplated the vascular repair assembly 100 could instead be advanced to a position downstream of the incompetent vein valve 12, or both.

FIG. 10B schematically depicts a cross section of the vessel 10 taken at line 10B-10B of FIG. 10A. As depicted, the vascular repair assembly 100 is advanced in a low-profile or un-deployed configuration such that the vascular repair assembly 100 may traverse the vessel 10 to the desired location. In some embodiments, the vascular repair assembly 100 may include a sheath (not shown) which may be advanced over the closure device 110 prior to deployment. When deployment is desired, the sheath may be withdrawn to expose the closure device 110. A sheath may be particularly beneficial when the one or more anchoring elements 130 including an adhesive 137 such as described above.

Still referring to FIG. 10B, the one or more anchoring elements 130 are illustrated as having a plurality of retention members 131. However, and as noted above, instead of or in addition to the plurality of retention members 131, an adhesive 137, such as described above, may be coated over the outer surface 114 of the tube 112 of the closure device 110.

In the depicted embodiment, the plurality of retention members 131 may each include, a base 132 and one or more retention legs 134, such as described herein, extending from the base 132. However, in the depicted embodiment, the base 132 defines a first base portion 133a coupled to the one or more retention legs 134 and a second base portion 133b coupled to the expansion device 104. The first base portion 133a and the second base portion 133b may be connected to one another via a break-away point 135. It is noted that while the one or more retention legs 134 are depicted external to the tube 112, in some embodiments, and as described herein, the one or more retention legs 134 may be substantially within the tube 112 when within the low-profile delivery configuration.

Referring now to FIG. 10C, the closure device 110 is deployed to the expanded tissue-engaging configuration. In particular, the expansion device 104 is expanded, thereby translating the expansion force to the closure device 110 and the plurality of retention members 131. That is, expansion of the expansion device 104 causes the tube 112 to circumferentially contact the vessel wall 11 and the plurality of retention members 131 to pierce the vessel wall 11. In some embodiments, such as where the one or more anchor elements 130 includes an adhesive 137, mere contact or contact for a sufficient time between the vessel wall 11 and the adhesive 137 positioned on the outer surface 114 134 of the tube 112 may anchor the closure device 110 to the vessel wall 11.

Referring now to FIG. 10D, once anchoring to the vessel wall 11 is completed, the expansion device 104 may be retracted back to the low-profile delivery configuration. In the illustrated embodiment, upon retraction, the second base portion 133b may be broken away from the first base portion 133a portion at the breakaway point 135 thereby leaving the first base portion 133a and the one or more retention legs 134 anchoring the closure device 110 to the vessel wall 11. By the second portion 133b being broken or detached from the first base portion 133a there may be less obstruction within the flow path of the vessel 10 through the tube 112. After separation, the catheter 102 and the expansion device 104 may then be withdrawn from the vessel 10.

Referring now to FIG. 10E, the closure device 110 is retracted back to a smaller diameter thereby drawing the vessel 10 to a retracted diameter. As noted above, the tube 112 of the closure device 110 is elastically deformable. Accordingly, once the expandable device 104 is retracted, the tube 112 of the closure device 110 may naturally retract back to a retracted diameter, which may be sized so as to pull the incompetent leaflets 14a, 14b of the vein valve 12 back together. Though the closure device 110 may be naturally retractable to smaller diameter, in some embodiments, the retraction of the expansion device 104 may pull the closure device 110 to the retracted diameter and thereafter, the expansion device 104 may be detached or removed from the closure device 110. It is noted that after retraction, the ECM of the tube 112 may remain within the vessel and used as a scaffold for vessel cells to populate.

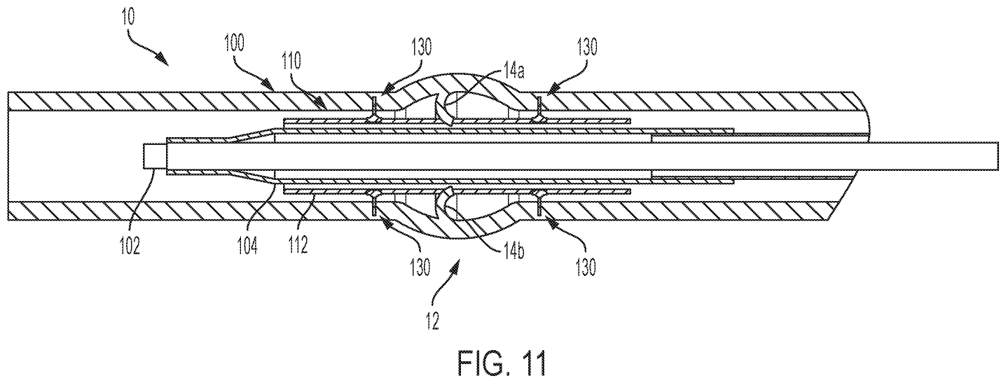

Referring now to FIG. 11 a vascular repair assembly 100, such as described herein, may be inserted through the incompetent valve 12 so as to longitudinally span the incompetent valve 12. In such embodiments, openings may be formed in the tube 112 to allow for the leaflets 14a, 14b to extend through or into the tube 112. In such embodiments, a portion of the plurality of anchoring elements 130 may be positioned upstream of the incompetent valve 12 and a second portion of the plurality of anchoring elements 130 may be positioned downstream of the incompetent vein valve 12 such that the tube 112 may be anchored to the vessel 10 at a position both upstream and downstream of the incompetent vein valve 12. Accordingly, the vessel 10 may be pulled to a narrower diameter both upstream of the incompetent vein valve 12 and downstream of the incompetent vein valve 12. This may lead to a more consistent diameter through the valve 12.

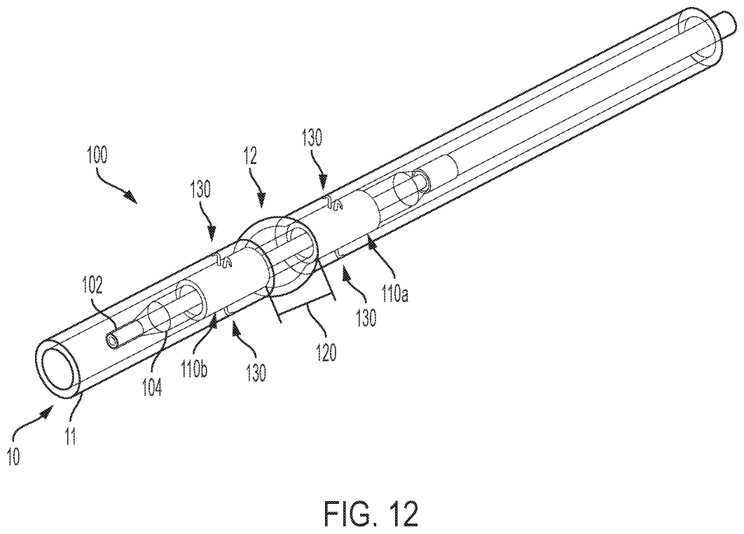

Referring now to FIG. 12, in some embodiments, a vascular repair assembly 100 may include a first closure device 110a deployed upstream of the incompetent valve 12 and a second closure device 110b deployed downstream of the incompetent valve 12. The first and second closure devices 110a, 110b may be substantially similar to those described above. The first closure device 110a and the second closure device 110b may be separate and distinct from one another. In some embodiments, a gap 120 may space the first closure device 110a from the second closure device 110b thereby separating the first closure device 110a from the second closure device 110b. In some embodiments, the vascular repair assembly 100 may simultaneously expand the first closure device 110a and second closure device 110b with the same expansion device 104. However, in other embodiments, the vascular repair assembly 100 may include a first expansion device for expanding the first closure device 110a and a second expansion device for expanding the second closure device 110b. Similar to the embodiment illustrated in FIG. 12, the vascular repair assembly 100 may be advanced such that the first closure device 110a is positioned upstream of the incompetent valve 12 and the second closure device 110b is positioned downstream of the incompetent valve 12. Accordingly, and similarly to the embodiment described in FIG. 11, the vessel 10 may be pulled to a narrower diameter both upstream of the incompetent vein valve 12 and downstream of the incompetent vein valve 12. This may lead to a more consistent diameter through the valve 12.

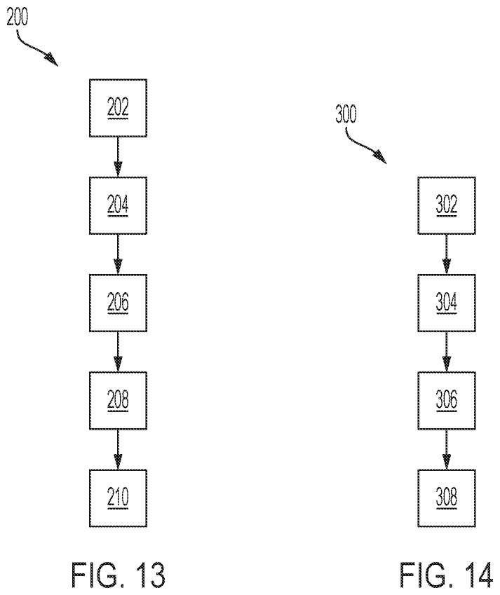

Referring now to FIG. 13, a flow chart depicting a method 200 of treating an incompetent vessel 10 is schematically depicted. It is noted that a greater or fewer number of steps may be included, in any order, without departing from the scope of the present disclosure. At step 202 the method 200 includes advancing the vascular repair assembly 100 so as to position one or more closure devices at a position proximate or adjacent (e.g., upstream, downstream, or both) to an incompetent valve 12 or other desired position within a vessel 10. As illustrated in FIG. 10A, the vascular repair assembly 100 may be advanced to a position upstream of the incompetent vessel 10. However, as illustrated in FIGS. 12 and 13 in some embodiments, the vascular repair assembly 100 may be advanced such as to be positioned both upstream and downstream of the incompetent vessel 10. In yet further embodiments, it is contemplated that the vascular repair assembly 100 may only be advanced to a position downstream of the incompetent vessel 10. At step 204, once in the desired position, the expansion device 104 may be expanded to place the closure device 110 (or closure devices) into circumferential contact with the vessel wall 11 of the vessel 10. At step 206, the method 200 includes anchoring the closure device 110 to the vessel wall 11. For example, and as described above, the closure device 110 may be adhered to the vessel 10, anchored to the vessel 10 via a plurality of retention members 131, such as described herein, or any combination thereof. At step 208, after anchoring the closure device 110 to the vessel 10, the method 200 includes retracting the expansion device 104 a low-profile orientation, such as illustrated in FIG. 10D. The catheter 102 and the expansion device 104 may be removed from the vessel 10 leaving the closure device 110 attached to the vessel 10, as illustrated in FIGS. 10D and 10E. At step 210, the expansion device 104 may retract to a retracted configuration, such as illustrated in FIG. 10E, thereby pulling the leaflets 14a, 14b of the incompetent vein valve 12 back together.

Referring now to FIG. 14, a flow chart depicting a method 300 of assembling a vascular repair assembly 100 is generally depicted. It is noted that a greater or fewer number of steps may be included without departing from the scope of the present disclosure. The method 300 may include, at step 302, providing a tube 112 of ECM including a plurality of elastin fibers 115 arranged to allow the tube 112 to diametrically expand and retract. In some embodiments, providing the tube 112 may include forming and/or growing the tube 112 of ECM such as in a laboratory setting. At step 304, the tube 112 may be subject to one or more sterilization and/or disinfection procedures such as ethylene oxide sterilization, peracetic acid disinfection, electron beam irradiation sterilization, plasma sterilization, etc., or any combination thereof. At step 306, the method 300 may include mounting the tube 112 may to an expansion device 104 such as illustrated in FIG. 9, 10A, 11, or 12. As noted herein the expansion device 104 itself may be mounted to or form part of a catheter 102. As further described above, the expansion device 104 may be radially expandable about the catheter 102 so as to circumferentially expand the closure device 110 mounted thereto. In some embodiments, and as illustrated in FIG. 12, a first closure device 110a may be mounted to the expansion device 104 and a second closure device 110b the same or a second expansion device 104 positioned on or otherwise part of the catheter 102. At step 308, the method 300 may include assembling one or more anchor elements 130 to the tube 112 of the closure device 110. For example, and as described in greater detail above, the one or more anchor elements 130 may include an adhesive 137 coated onto the outer surface 114 of the tube 112. In addition to or in lieu of the adhesive 137, the one or more anchor elements 130 may include a plurality of retention members 131, which may be may be mounted to the tube 112 and/or the expandable device 104 as described herein. In some embodiments, where the one or more anchoring elements 130 are mounted to the expansion device 104, one or more guide paths 118 may be formed in the tube 112 for receiving one or more retention legs 134 of the plurality of retention members 131, such as described above. Such guide paths 118 may be formed via cuts, drilling, laser ablation, or the like.

In some embodiments, prior to use or shipment, the vascular repair assembly 100 may be subject to sterilization and/or disinfection, for example, using ethylene oxide sterilization, peracetic acid disinfection, electron beam irradiation sterilization, plasma sterilization, etc., or any combination thereof. In some embodiments, prior to assembly to the expansion device 104 the closure device 110 may first be subject to peracetic acid disinfection and/or one or more other sterilization procedures as noted above. Then, once assembled or in a disassembled state, the vascular repair assembly 100 may then be collectively subject to one or more sterilization procedures as noted above. In some embodiments, the vascular repair assembly 100 may be packages in a sterilizable packaging, which may also be subject to one or more disinfection and/or sterilization procedures as noted above.

In various embodiments, the vascular repair assembly 100 may be provided to a user as a kit, which may be assembled by the user. In some embodiments, the vascular repair assembly 100 may be provided fully assembled.

Embodiments can be described with reference to the following numerical clause:

1. A closure device for repairing a vein valve insufficiency, the closure device comprising: a tube formed of extracellular matrix, comprising elastin fibers, wherein the tube is radially expandable from a retracted configuration to an expanded configuration, wherein the tube is naturally biased to the retracted configuration; and one or more anchoring elements configured to anchor the tube to a vessel wall of a vessel upon expansion of the tube to the expanded configuration wherein the tube circumferentially contacts the vessel wall of the vessel and is anchored thereto by the one or more anchoring elements; and wherein retraction of the tube to the retracted configuration draws the vessel wall of the vessel radially inward to repair the vein valve insufficiency.

2. The closure device of clause 1, wherein the one or more anchoring elements comprise a cell adhesion molecule coated on an outside surface of the tube, wherein the cell adhesion molecule chemically reacts to adhere the tube to the vessel wall of the vessel.

3. The closure device of any preceding clause, wherein the one or more anchoring elements comprise a plurality of retention members configured to pierce the vessel wall of the vessel.

4. The closure device of any preceding clause, wherein the plurality of retention members comprise one or more retention legs extending from a base that is configured to engage the tube, wherein expansion of the tube causes the one or more retention legs to pierce the vessel wall of the vessel.

5. The closure device of any preceding clause, wherein the one or more retention legs comprise a first retention leg and a second retention leg, wherein the first retention leg and the second retention leg diverge from one another as the first retention leg and the second retention leg are advanced through the vessel wall of the vessel.

6. The closure device of any preceding clause, wherein the one or more retention legs are arranged against a surface of the tube prior to expansion of the tube, and where expansion of the tube causes the one or more retention legs to extend away from the surface of the tube.

7. The closure device of any preceding clause, wherein the one or more anchoring elements comprise a plurality of retention members comprising: a retention leg having a needle-like tip; a base coupled to a proximal end of the retention leg; and an expandable shield coupled to the needle-like tip, wherein the needle-like tip and the expandable shield are configured to be advanced through the vessel wall of the vessel upon expansion of the tube to the expanded configuration, such that the vessel wall of the vessel becomes positioned between the base and the expandable shield, wherein the expandable shield is configured to be compressed to be advanced through the vessel wall of the vessel and is configured to expand after passing through the vessel wall of the vessel to trap the vessel wall of the vessel between the base and the expandable shield.

8. The closure device of any preceding clause, wherein the plurality of retention members are formed from extracellular matrix.

9. A vascular repair assembly, comprising: an expansion device, wherein the expansion device is configured to radially expand; and a closure device removably mounted to the expansion device for delivery into a vessel, comprising: a tube formed of extracellular matrix, comprising elastin fibers, wherein the expansion device is positioned within a lumen of the tube and the tube is radially expandable from a retracted configuration to an expanded configuration in response to expansion of the expansion device, wherein the tube is naturally biased to the retracted configuration upon removal of the expansion device, and one or more anchoring elements configured to anchor the tube to a vessel wall of the vessel upon expansion of the tube to the expanded configuration wherein the tube circumferentially contacts the vessel wall of the vessel and is anchored thereto by the one or more anchoring elements, and wherein retraction of the expansion device allows the tube to retract to the retracted configuration thereby drawing the vessel wall of the vessel radially inward as the tube retracts to the retracted configuration.

10. The vascular repair assembly of any preceding clause, wherein the one or more anchoring elements comprise a cell adhesion molecule coated on an outside surface of the tube, wherein the cell adhesion molecule chemically reacts to adhere the tube to the vessel wall of the vessel.

11. The vascular repair assembly of any preceding clause, wherein the one or more anchoring elements comprise a plurality of retention members configured to engage the tube and configured to pierce the vessel wall of the vessel.

12. The vascular repair assembly of any preceding clause, wherein the plurality of retention members each comprise one or more retention legs extending from a base coupled to the tube, wherein expansion of the tube causes the one or more retention legs to pierce the vessel wall of the vessel.

13. The vascular repair assembly of any preceding clause, wherein the one or more retention legs comprise a first retention leg and a second retention leg, wherein the first retention leg and the second retention leg diverge from one another as the first retention leg and the second retention leg are advanced through the vessel wall of the vessel.

14. The vascular repair assembly of any preceding clause, wherein the one or more retention legs are arranged against a surface of the tube prior to expansion of the tube, and where expansion of the tube causes the one or more retention legs to extend away from the surface of the tube.

15. The vascular repair assembly of any preceding clause, wherein the one or more anchoring elements comprise a plurality of retention members comprising: a retention leg having a needle-like tip; a base coupled to a proximal end of the retention leg; and an expandable shield coupled to the needle-like tip, wherein the needle-like tip and the expandable shield are configured to be advanced through the vessel wall of the vessel upon expansion of the tube to the expanded configuration, such that the vessel wall of the vessel becomes positioned between the base and the expandable shield, wherein the expandable shield is configured to be compressed to be advanced through the vessel wall of the vessel and is configured to expand after passing through the vessel wall of the vessel to trap the vessel wall of the vessel between the base and the expandable shield.

16. The vascular repair assembly of any preceding clause, wherein the plurality of retention members are formed from extracellular matrix.

17. The vascular repair assembly of any preceding clause, wherein: the one or more anchoring elements comprise a plurality of retention members each comprising a base coupled to the expansion device; the tube defines one or more guide paths; and a retention member of the plurality of retention members pass through the one or more guide paths in response to expansion of the expansion device.

18. The vascular repair assembly of any preceding clause, wherein the closure device is a first closure device and the vascular repair assembly further comprises a second closure device removably mounted to the expansion device and longitudinally spaced from the closure device such that a gap is positioned between the first closure device and the second closure device.

19. The vascular repair assembly of any preceding clause, wherein: the one or more anchoring elements comprise a base coupled to the expansion device; and retraction of the expansion device disconnects the one or more anchoring elements from the expansion device.

20. The vascular repair assembly of any preceding clause, wherein the expansion device is a balloon.

21. A method of repairing a vein valve insufficiency, the method comprising: advancing a closure device mounted to an expansion device through a vessel to a position adjacent a target vein valve, wherein the closure device comprises a tube and one or more anchoring elements, the tube being formed of extracellular matrix comprising elastin fibers; expanding the tube to an expanded configuration with the expansion device such that the tube is in circumferential contact with a vessel wall of the vessel and the one or more anchoring elements anchor the tube to the vessel wall of the vessel; and retracting the expansion device such that the tube retracts to a retracted configuration, wherein the tube is naturally biased to the retracted configuration.

22. The method of any preceding clause, wherein the one or more anchoring elements comprise a cell adhesion molecule coated on an outside surface of the tube, wherein the cell adhesion molecule chemically reacts to adhere the tube to the vessel wall of the vessel.

23. The method of any preceding clause, wherein the one or more anchoring elements comprise one or more retention legs extending from a base configured to engage the tube, wherein expanding the tube causes the one or more retention legs to pierce the vessel wall of the vessel.

24. The method of any preceding clause, wherein the one or more retention legs are arranged against a surface of the tube prior to expanding of the tube, and wherein expanding of the tube causes the one or more retention legs to extend away from the surface of the tube.

25. The method of any preceding clause, further comprising advancing a second closure device mounted to the expansion device through the vessel to a position adjacent the target vein valve, wherein the one or more anchoring elements of the second closure device anchor the second closure device to the vessel upon expansion of the tube into radial contact with the vessel wall of the vessel.

26. A method of assembling a vascular repair assembly, the method comprising: assembling a closure device comprising a tube formed of extracellular matrix including a plurality of elastin fibers on an expansion device, wherein the tube is radially expandable to an expanded configuration and is naturally retractable to a retracted configuration, wherein the closure device comprises one or more anchoring elements configured to anchor the closure device to a vessel wall of a vessel upon expansion of the tube to the expanded configuration.

27. The method of any preceding clause further comprising: assembling the one or more anchoring elements within the vascular repair assembly.

28. The method of any preceding clause, wherein assembly the one or more anchoring elements within the vascular repair assembly comprises coating an outer surface of the tube with an adhesive.

29. The method of any preceding clause, wherein assembly the one or more anchoring elements within the vascular repair assembly comprises coupling a plurality of retention members to at least one of the tube and the expansion device.

30. The method of any preceding clause, wherein the plurality of retention members each comprise a base configured to be coupled to the at least one of the tube and the expansion device and one or more retention legs extending from the base.

31. The method of any preceding clause, wherein the expansion device is mounted to a catheter and is configured to circumferentially expand around the catheter.

32. The method of any preceding clause, wherein the closure device is a first closure device and the method further comprises mounting a second closure device to the expansion device, such that the second closure device is longitudinally spaced and separate from the first closure device.

It should now be understood that embodiments of the present disclosure are directed to closure devices, vascular repair assemblies, and methods for repairing a vein valve insufficiency. For example, a closure device may include a tube formed of, for example extracellular matrix, comprising elastin fibers, wherein the tube is radially expandable from a retracted configuration to an expanded configuration. The tube is formed so as to be naturally biased to the retracted configuration. One or more anchoring elements are configured to anchor the tube to a vessel wall of a vessel (e.g., a vein or other bodily lumen) upon expansion of the tube to the expanded configuration. When expanded, the tube circumferentially contacts the vessel wall of the vessel and is anchored thereto by the one or more anchoring elements. The tube may be retracted to the retracted configuration thereby drawing the vessel wall of the vessel radially inward to repair the vein valve insufficiency. Accordingly, the tube may pull the vessel wall back to an operable diameter to restore natural function to the vessel valve.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A closure device for repairing a vein valve insufficiency, the closure device comprising:
a tube formed of extracellular matrix, comprising elastin fibers, wherein the tube is radially expandable from a retracted configuration to an expanded configuration, wherein the tube is naturally biased to the retracted configuration; and
one or more anchoring elements configured to anchor the tube to a vessel wall of a vessel upon expansion of the tube to the expanded configuration wherein the tube circumferentially contacts the vessel wall of the vessel and is anchored thereto by the one or more anchoring elements; and
wherein retraction of the tube to the retracted configuration draws the vessel wall of the vessel radially inward to repair the vein valve insufficiency.

2. The closure device of claim 1, wherein the one or more anchoring elements comprise a cell adhesion molecule coated on an outside surface of the tube, wherein the cell adhesion molecule chemically reacts to adhere the tube to the vessel wall of the vessel.

3. The closure device of claim 1, wherein the one or more anchoring elements comprise a plurality of retention members configured to pierce the vessel wall of the vessel.

4. The closure device of claim 3, wherein the plurality of retention members comprise one or more retention legs extending from a base configured to be engaged with the tube, wherein expansion of the tube causes the one or more retention legs to pierce the vessel wall of the vessel.

5. The closure device of claim 4, wherein the one or more retention legs comprise a first retention leg and a second retention leg, wherein the first retention leg and the second retention leg diverge from one another as the first retention leg and the second retention leg are advanced through the vessel wall of the vessel.

6. The closure device of claim 4, wherein the one or more retention legs are arranged against a surface of the tube prior to expansion of the tube, and where expansion of the tube causes the one or more retention legs to extend away from the surface of the tube.

7. The closure device of claim 1, wherein the one or more anchoring elements comprise a plurality of retention members comprising:
a retention leg having a needle-like tip;
a base coupled to a proximal end of the retention leg; and
an expandable shield coupled to the needle-like tip,
wherein the needle-like tip and the expandable shield are configured to be advanced through the vessel wall of the vessel upon expansion of the tube to the expanded configuration, such that the vessel wall of the vessel becomes positioned between the base and the expandable shield, wherein the expandable shield is configured to be compressed to be advanced through the vessel wall of the vessel and is configured to expand after passing through the vessel wall of the vessel to trap the vessel wall of the vessel between the base and the expandable shield.

8. The closure device of claim 4, wherein the plurality of retention members are formed from extracellular matrix.

9. A vascular repair assembly, comprising:
an expansion device, wherein the expansion device is configured to radially expand; and
a closure device removably mounted to the expansion device for delivery into a vessel, comprising:
a tube formed of extracellular matrix, comprising elastin fibers, wherein the expansion device is positioned within a lumen of the tube and the tube is radially expandable from a retracted configuration to an expanded configuration in response to expansion of the expansion device, wherein the tube is naturally biased to the retracted configuration upon removal of the expansion device, and
one or more anchoring elements configured to anchor the tube to a vessel wall of the vessel upon expansion of the tube to the expanded configuration wherein the tube circumferentially contacts the vessel wall of the vessel and is anchored thereto by the one or more anchoring elements, and
wherein retraction of the expansion device allows the tube to retract to the retracted configuration thereby drawing the vessel wall of the vessel radially inward as the tube retracts to the retracted configuration.

10. The vascular repair assembly of claim 9, wherein the one or more anchoring elements comprise a cell adhesion molecule coated on an outside surface of the tube, wherein the cell adhesion molecule chemically reacts to adhere the tube to the vessel wall of the vessel.

11. The vascular repair assembly of claim 9, wherein the one or more anchoring elements comprise a plurality of retention members configured to engage the tube and configured to pierce the vessel wall of the vessel.

12. The vascular repair assembly of claim 11, wherein the plurality of retention members each comprise one or more retention legs extending from a base coupled to the tube, wherein expansion of the tube causes the one or more retention legs to pierce the vessel wall of the vessel.

13. The vascular repair assembly of claim 12, wherein the one or more retention legs comprise a first retention leg and a second retention leg, wherein the first retention leg and the second retention leg diverge from one another as the first retention leg and the second retention leg are advanced through the vessel wall of the vessel.

14. The vascular repair assembly of claim 12, wherein the one or more retention legs are arranged against a surface of the tube prior to expansion of the tube, and where expansion of the tube causes the one or more retention legs to extend away from the surface of the tube.

15. The vascular repair assembly of claim 9, wherein the one or more anchoring elements comprise a plurality of retention members comprising:
a retention leg having a needle-like tip;
a base coupled to a proximal end of the retention leg; and
an expandable shield coupled to the needle-like tip,
wherein the needle-like tip and the expandable shield are configured to be advanced through the vessel wall of the vessel upon expansion of the tube to the expanded configuration, such that the vessel wall of the vessel becomes positioned between the base and the expandable shield, wherein the expandable shield is configured to be compressed to be advanced through the vessel wall of the vessel and is configured to expand after passing through the vessel wall of the vessel to trap the vessel wall of the vessel between the base and the expandable shield.

16. The vascular repair assembly of claim 11, wherein the plurality of retention members are formed from extracellular matrix.

17. The vascular repair assembly of claim 11, wherein:
the one or more anchoring elements comprise a plurality of retention members each comprising a base coupled to the expansion device;
the tube defines one or more guide paths; and
a retention member of the plurality of retention members pass through the one or more guide paths in response to expansion of the expansion device.

18. The vascular repair assembly of claim 9, wherein the closure device is a first closure device and the vascular repair assembly further comprises a second closure device removably mounted to the expansion device and longitudinally spaced from the closure device such that a gap is positioned between the first closure device and the second closure device.

19. The vascular repair assembly of claim 9, wherein:
the one or more anchoring elements comprise a base coupled to the expansion device; and
retraction of the expansion device disconnects the one or more anchoring elements from the expansion device.

20. The vascular repair assembly of claim 9, wherein the expansion device is a balloon.

21. A method of repairing a vein valve insufficiency, the method comprising:
advancing a closure device mounted to an expansion device through a vessel to a position adjacent a target vein valve, wherein the closure device comprises a tube and one or more anchoring elements, the tube being formed of extracellular matrix comprising elastin fibers;
expanding the tube to an expanded configuration with the expansion device such that the tube is in circumferential contact with a vessel wall of the vessel and the one or more anchoring elements anchor the tube to the vessel wall of the vessel; and
retracting the expansion device such that the tube retracts to a retracted configuration, wherein the tube is naturally biased to the retracted configuration.

22. The method of claim 21, wherein the one or more anchoring elements comprise a cell adhesion molecule coated on an outside surface of the tube, wherein the cell adhesion molecule chemically reacts to adhere the tube to the vessel wall of the vessel.

23. The method of claim 21, wherein the one or more anchoring elements comprise one or more retention legs extending from a base configured to engage the tube, wherein expanding the tube causes the one or more retention legs to pierce the vessel wall of the vessel.

24. The method of claim 23, wherein the one or more retention legs are arranged against a surface of the tube prior to expanding of the tube, and wherein expanding of the tube causes the one or more retention legs to extend away from the surface of the tube.

25. The method of claim 21, further comprising advancing a second closure device mounted to the expansion device through the vessel to a position adjacent the target vein valve, wherein the one or more anchoring elements of the second closure device anchor the second closure device to the vessel upon expansion of the tube into radial contact with the vessel wall of the vessel.

26. A method of assembling a vascular repair assembly, the method comprising:
assembling a closure device comprising a tube formed of extracellular matrix including a plurality of elastin fibers on an expansion device, wherein the tube is radially expandable to an expanded configuration and is naturally retractable to a retracted configuration, wherein the closure device comprises one or more anchoring elements configured to anchor the closure device to a vessel wall of a vessel upon expansion of the tube to the expanded configuration.

27. The method of claim 26 further comprising:
assembling the one or more anchoring elements within the vascular repair assembly.

28. The method of claim 27, wherein assembly the one or more anchoring elements within the vascular repair assembly comprises coating an outer surface of the tube with an adhesive.

29. The method of claim 27, wherein assembly the one or more anchoring elements within the vascular repair assembly comprises coupling a plurality of retention members to at least one of the tube and the expansion device.

30. The method of claim 29, wherein the plurality of retention members each comprise a base configured to be coupled to the at least one of the tube and the expansion device and one or more retention legs extending from the base.

31. The method of claim 29, wherein the expansion device is mounted to a catheter and is configured to radially expand around the catheter.

32. The method of claim 27, wherein the closure device is a first closure device and the method further comprises mounting a second closure device to the expansion device, such that the second closure device is longitudinally spaced and separate from the first closure device.
